(19) United States
(12) Patent Application Publication
(10) Pub. No.: US 2021/0245005 A1
(43) Pub. Date: Aug. 12, 2021

(54) IMPLEMENTATION OF MACHINE LEARNING FOR SKILL-IMPROVEMENT THROUGH CLOUD COMPUTING AND METHOD THEREFOR

(71) Applicant: FOCALTRON CORPORATION, Sunnyvale, CA (US)

(72) Inventors: YI-CHING PAO, SUNNYVALE, CA (US); JAMES J. PAO, SUNNYVALE, CA (US)

(21) Appl. No.: 17/075,371

(22) Filed: Oct. 20, 2020

Related U.S. Application Data
(60) Provisional application No. 62/909,622, filed on Oct. 2, 2019.

(51) Int. Cl. A63B 24/00; A63B 71/06; A63B 69/36; G06N 20/00

(52) U.S. Cl. CPC A63B 24/0006; G06F 16/9535; A63B 69/3623; G06N 20/00; A63B 2220/05; A63B 2071/0694; A63B 2071/063; A63B 2220/803; A63B 2220/806; A63B 2220/807; A63B 2225/20; A63B 2225/50; A63B 2024/0015; A63B 2024/0031; A63B 71/0622

(57) ABSTRACT

A method for generating feedback to a user practicing a skill comprises: providing a local platform for acquiring physical parameter data pertaining to motion and position of the user and motion and position of a golf club and a golf ball struck by the golf club during a golf swing by the user; transmitting via a network at least a portion of the physical parameter data of the motion and position of the golf club and the golf ball struck by the golf club during the golf swing and the physical parameter data associated with the motion and position of the user during the golf swing from the local platform to a machine learning analysis engine as input information; entering the input information into a machine learning model, the machine learning model having a set of rules and statistical techniques to learn patterns from the input data, and a model which is trained by using evolving training sets, wherein an initial training sets is formed from selected professional golf players physical and swing characteristics and are classified and used to train the machine learning model and resulting learned weighting factors are feedback and used to refine a model prediction, the machine learning model determining a user's skill deficiencies and providing correction suggestions; and providing a correction suggestion to the user.

### RELATED APPLICATIONS

This patent application is related to U.S. Provisional Application No. 62/909,622 filed Oct. 2, 2019, entitled “IMPLEMENTATION OF MACHINE LEARNING FOR SKILL-IMPROVEMENT THROUGH CLOUD COMPUTING” in the names of Yi-Ching Pao and James J. Pao, and which is incorporated herein by reference in its entirety. The present patent application claims the benefit under 35 U.S.C § 119(e).

### TECHNICAL FIELD

The present application generally relates to a sensory platform for monitoring and recording device used to monitor and record movement of a user and, more particularly, to a sensory platform for monitoring and recording device used to monitor and record movement of a user which uses machine learning to optimize and improve the accuracy of a knowledge-based instruction engine.

### BACKGROUND

There are situations, especially in motion intensive sports such as golf, baseball, soccer, tennis, archery, shooting and the like, where the movements of objects or motions of players may be paramount to the game. In these sports, a player may constantly strive to improve or perfect such movements. For example, a player may wish to not only understand and seek instant feedback regarding the motion of any pertinent play objects such as balls, rackets, and clubs, but to further seek identification and improvement of deficiencies and mistakes in the player's swing or motion technique, and to leverage on an implemented “machine learning” system to identify and pin point the problematic areas for key skill improvements.

In Applicant's recently granted patent, U.S. Pat. No. 10,339,821 B2, an example of a sensory platform may be disclosed which utilizes video images and/or shot data from sensory platforms to capture and send captured data and images through the Internet for analysis and interpretation. While the sensory platform disclosed in U.S. Pat. No. 10,339,821 B2 may help to identify and pin point the problematic areas for the player, it does not have the ability to optimize and improve the correctness and the accuracy of the knowledge-based instruction engine.

Machine learning is an application of artificial intelligence (AI) that may provide systems the ability to automatically learn and improve from experience without being explicitly programmed. Machine learning focuses on the development of computer programs that can access data and use it learn for themselves. Thus, the ability to “machine learning” may allow one to optimize and improve the correctness and the accuracy of knowledge-based instruction engine may be improved. Machine learning may typically be accomplished by an algorithm consisting of a set of rules and statistical techniques used to learn patterns from data, and a model which is trained by using the algorithm. Such specialized “machine learning” algorithm and model, based on (1) players' physical profiles, (2) captured shot data, and (3) processed player's swing and posture images for players' skill improvement, may be suitable primarily for online golf instruction and coaching session which can be scaled up to accommodate multiple players at any given time and from anywhere in the world.

Therefore, it would be desirable to provide a system and method that overcome the above problems. The system and method would use “machine learning” to optimize and improve the correctness and the accuracy of knowledge-based instruction engine

### SUMMARY

In accordance with one embodiment, a method for generating feedback to a user practicing a skill is disclosed. The method comprising: providing a local platform for acquiring physical parameter data pertaining to motion and position of the user and motion and position of a golf club and a golf ball struck by the golf club during a golf swing by the user; transmitting via a network at least a portion of the physical parameter data of the motion and position of the golf club and the golf ball struck by the golf club during the golf swing and the physical parameter data associated with the motion and position of the user during the golf swing from the local platform to a machine learning analysis engine as input information; entering the input information into a machine learning model, the machine learning model having a set of rules and statistical techniques to learn patterns from the input data, and a model which is trained by using evolving training sets, wherein an initial training sets is formed from selected professional golf players physical and swing characteristics and are classified and used to train the machine learning model, and resulting learned weighting factors are feedback and used to refine a model prediction, the machine learning model determining a user's skill deficiencies and providing correction suggestions; and providing a correction suggestion to the user.

In accordance with one embodiment, a method for generating feedback to a user practicing a skill is disclosed. The method comprising: providing a local platform for acquiring physical parameter data pertaining to motion and position of the user and motion and position of a golf club and a golf ball struck by the golf club during a golf swing by the user; transmitting via a network the physical parameter data of the motion and position of the golf club and the golf ball struck by the golf club during the golf swing and the physical parameter data associated with the motion and position of the user during the golf swing recorded by the local platform to a machine learning analysis engine as input information; and entering the input information into a machine learning model, the machine learning model having a set of rules and statistical techniques to learn patterns from the input data, and a model which is trained by using evolving training sets, wherein an initial training sets is formed from selected professional golf players physical and swing characteristics and are classified and used to train the machine learning model, and resulting learned weighting factors are feedback and used to refine a model prediction, the machine learning model determining a user's skill deficiencies and providing correction suggestions.

### BRIEF DESCRIPTION OF THE DRAWINGS

The present application is further detailed with respect to the following drawings. These figures are not intended to limit the scope of the present application but rather illustrate certain attributes thereof. The same reference numbers will be used throughout the drawings to refer to the same or like parts.

FIG. 1 is a block diagram of an exemplary embodiment of a network system of individual sensory platforms and a designated knowledge based “machine learning” unit in accordance with one embodiment of the present invention;

FIG. 2 is a block diagram of an exemplary embodiment of a computing device/server used in the network system of FIG. 1 in accordance with one embodiment of the present invention;

FIG. 3A show an exemplary embodiment of a user's physical profile data used as an input category for the machine learning process to provide online instruction in accordance with one embodiment of the present invention;

FIG. 3B show an exemplary embodiment of a shot captured data by a sensory platform used as an input category for the machine learning process to provide online instruction in accordance with one embodiment of the present invention;

FIG. 3C show an exemplary embodiment of processed video swing images and relevant postures of the player during play used as an input category for the machine learning process to provide online instruction in accordance with one embodiment of the present invention;

FIG. 4 is shows exemplary embodiments of nine basic projectiles, such as a golf ball, flight patterns and the most common mistakes made by a player which result in a non-desired flight pattern, the machine leaning process leverages on the training sets which initially are derived from professional players as the baseline, and player's accumulated shot and swing image data to further train the model for individualized improvement in accordance with one embodiment of the present invention;

FIG. 5 is a block diagram of an exemplary embodiment of the operation of the machine learning unit in accordance with one embodiment of the present invention; and

FIG. 6 shows an exemplary embodiment of process flow of steps for implementing the machine learning unit in accordance with one embodiment of the present invention.

### DESCRIPTION OF THE APPLICATION

The description set forth below in connection with the appended drawings is intended as a description of presently preferred embodiments of the disclosure and is not intended to represent the only forms in which the present disclosure can be constructed and/or utilized. The description sets forth the functions and the sequence of steps for constructing and operating the disclosure in connection with the illustrated embodiments. It is to be understood, however, that the same or equivalent functions and sequences can be accomplished by different embodiments that are also intended to be encompassed within the spirit and scope of this disclosure.

Embodiments of the exemplary sensory platform network may utilize a knowledge-based machine learning instruction unit. The sensory platform units may be capable of performing object physical parametric data and video image acquisition and processing. This information may be transmitted over a network connection to the knowledge-based machine learning instruction unit. The knowledge-based machine learning instruction unit may continually learn over time, rendering the most relevant and accurate play skill improvement instructions through cloud computation. While the exemplary sensory platform network may be used to improve any type of activity, an activity of particular interest is the sport of golf. For this reason, golf will be used as the primary example herein although it should be understood that the embodiments disclosed may be used for any type of activity and is not limited only to golf.

Generally speaking, a well performed golf strike may consists of a multitude of factors. For example, considerations should be given to the playing course's topographic qualities, as well as spatial layouts and weather conditions. Beyond these, a golf player should use situationally appropriate gear, such as the correct club. Once appropriately equipped, the player may have to perform a golf club swing with the golf club striking the golf ball to or towards the target. This strike may be an exceptionally complex compound action, composed of large actions and smaller more subtle actions. In this example, both the golf ball and the club represent play objects for data collection and analysis purposes.

Player technique considerations include for example feet positioning relative to one another and to a target line, shoulder movement during the swing, body positioning relative to the ball, lateral head and body movements during the swing, arm positioning during the swing, forearm and upper-arm angle during the swing, torso rotation during the swing, pivoting of the feet during the swing, rotation of the club during the swing, club face angle during contact with the ball, arm and club positioning immediately before and after the swing, and velocity of the swing.

The factors listed, along with other secondary factors, can affect for example the quality and effectiveness of the swing and consequently the quality of the shot. The measured ball speed, trajectory, and spin can all be affected through the use of localized individual sensory platform, leading to a large range of shot effectiveness, given a certain game situation. Therefore, it is of extreme interest to develop an ability to determine any flaws in technique in order to improve and perfect the skills that may be necessary to consistently achieve a proper swing and strike which may allow the player to place the ball as close to the target as physically and situationally possible.

In order to achieve real time computation and instant feedback, including retrieving corrective instructions/feedback, the same inventors of this current application filed and were granted U.S. Pat. No. 10,339,821 B2 on Jul. 2, 2019. U.S. Pat. No. 10,339,821 B2 implements in one or more embodiments, a local platform that is capable of (1) measuring all physical parameters relating to the players' skill assessment (including physical parameters pertaining to the play objects and to one or more body parts of the player for example), (2) calculating and converting all measured physical parameters and/or images into predetermined and simplified numerical data of a pre-defined format, and (3) communicating with designated cloud servers via a network such as the internet where the knowledge engine and databases reside with the network service provider.

As mentioned, the measured physical parameters may include but not limited to those pertaining to played objects such as ball, club, racket, and body motions, speeds, angles, trajectories, and video images. One physical sensory platform which is capable of capturing all physical parametric data is an L-shaped optical laser and sensor net. This optical net may be constructed in a variety of ways, including an “L” frame wherein the physical frame of a lengthwise base is placed upon the ground and a slightly angled post may be joined together and the emitters may be placed at each end, with a corresponding array of detectors along the base and the post. This L-shaped optical net is patented by the same inventors as the present application under U.S. Pat. No. 6,302,802 B1, granted on Oct. 16, 2001. However, it should be noted that other types of sensory platforms may be used without departing from the spirit and scope of the present invention.

The current invention extends both U.S. Pat. Nos. 6,302,802 B1 and 10,339,821 B2 by adding and including new features of a neural network-based machine learning algorithm and model with cumulative and evolving training sets. In accordance with one embodiment, the neural network-based machine learning algorithm and model may use three categories of input data, namely players' physical profiles, captured shot data, and swing images plus an initial training set derived from professional players.

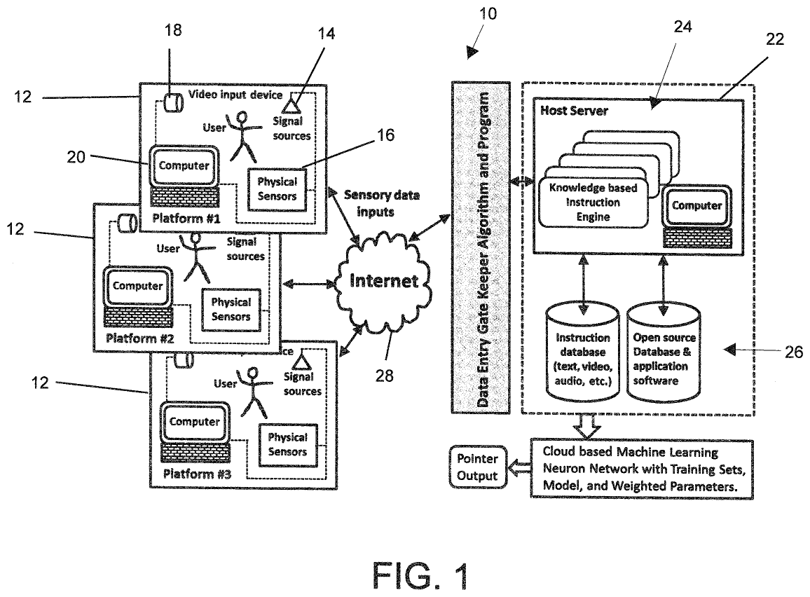

Referring to FIG. 1, one embodiment of a sensory platform networked system 10 (hereinafter system 10) may be seen. The system 10 may have a plurality of sensory and imaging platforms 12. The sensory and imaging platforms 12 may be used to monitor and record a user's movement, in accordance with one embodiment, the sensory and imaging platforms 12 may be capable of (1) measuring all physical parameters relating to a user's skill assessment (including physical parameters pertaining to the play objects and to one or more body parts of the user for example), (2) calculating and converting all measured physical parameters and/or images into predetermined and simplified numerical data of a pre-defined format, and (3) communicating with designated cloud servers via a network such as the internet where a knowledge engine and databases reside with the network service provider. The sensory and imaging platforms 12 may include a combination of Internet connecting devices such as: light or free space signal sources 14, physical sensing devices 16, video/image capturing devices 18, and local computing devices 20. The main function of the sensory and imaging platforms 12 may be to capture the required physical parameters associated with the motion of the play objects and motion image of the players, and then perform local computation, analysis, and data conversion of parametric values acquired. As stated above, the sensory and imaging platforms 12 may be similar to that disclosed in U.S. Pat. Nos. 6,302,802 B1 and 10,339,21 B2 both of which are incorporated by reference.

The system 10 may have a server 22. The server 22 may be used to host an analysis engine 24. The analysis engine 24 may be used to receive the data monitored and recorded by the sensory and imaging platforms 12 and to generate feedback to correct and improve the motion monitored. The analysis engine 24 may have a machine learning module 24A. The machine learning module 24A may be a knowledge engine that includes and/or has access to knowledge-based instruction databases 26, allowing for a combination of expert-developed, hard-coded feedback roles and machine learning predictive models to deliver real-time instructional feedback through a variety of modalities including text, graphs, audio, video, or some combination of all. One design of this feedback system could be a knowledge-based engine instruction database that analyzes (cleans, categorizes, quantifies, etc.) the shot data and captured images and perform feature engineering to provide inputs to the machine learning model with features expected to have strong predictive values. As machine learning models rely upon large datasets for generalization ability, the machine learning models and feature engineering can be updated and improved with the accumulation of more labeled data, which may either be engineered data or test-time data whose ground-truth is verified by an expert after collection.

The sensory and imaging platforms 12 may format the processed results into a streamlined and simplified data stream to communicate with the analysis engine 24 through network connections. The sensory and imaging platforms 12 may communicate with the analysis engine 24 hosted on the server 22 through an application programming interfaces (API). The sensory and imaging platforms 12 may communicate with the analysis engine 22 through a network 28. The network 28 may be network such as the World Wide Web.

Once analysis is done by the analysis engine 24, the initial output of the analysis is then fed into a machine learning model 24A of the analysis engine 24 with preliminary training sets and weighted factors for decision optimization and determination. Over time and continuous practice this process further train and update the training sets and model of a specific player based on the accumulated shot and image data which keeps improving the relevance and accuracy in a progressive way of the skill improvement instructions given by the machine learning system accordingly.

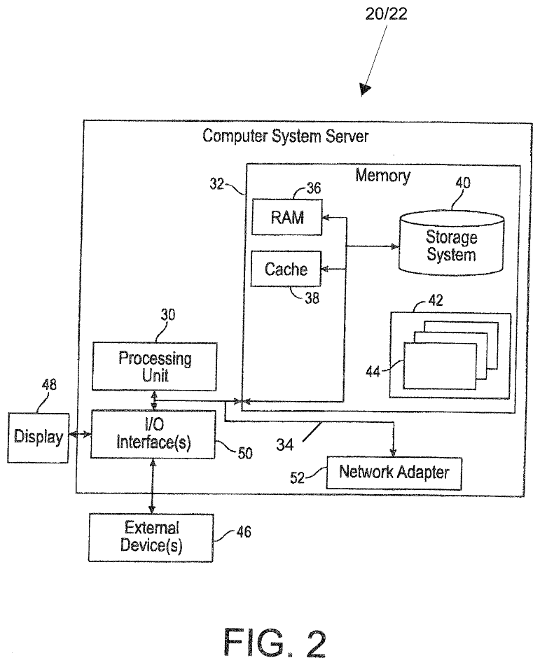

Referring to FIG. 2, the computing devices 20 and server 22 (hereinafter computing devices 20) may be described in more detail in terms of the machine elements that provide functionality to the systems and methods disclosed herein. The components of the computing devices 20 may include, but are not limited to, one or more processors or processing units 30, a system memory 32, and a system bus 34 that couples various system components including the system memory 32 to the processor 30. The computing devices 20 may typically include a variety of computer system readable media. Such media could be chosen from any available media that is accessible by the computing devices 20, including non-transitory, volatile and non-volatile media, removable and non-removable media. The system memory 32 could include one or more computer system readable media in the form of volatile memory, such as a random-access memory (RAM) 36 and/or a cache memory 38. By way of example only, a storage system 40 may be provided for reading from and writing to a non-removable, non-volatile magnetic media device typically called a “hard drive”.

The system memory 32 may include at least one program product/utility 42 having a set (e.g., at least one) of program modules 44 that may be configured to carry out the functions of embodiments of the invention. The program modules 44 may include, but is not limited to, an operating system, one or more application programs, other program modules, and program data. Each of the operating systems, one or more application programs, other program modules, and program data or some combination thereof, may include an implementation of a networking environment.

The computing device 20 may communicate with one or more external devices 46 such as a keyboard, a pointing device, a display 48, and/or any similar devices (e.g., network card, modern, etc.) that enable the computing device 20 to communicate with the server 24 (FIG. 1). Such communication may occur via Input/Output (I/O) interfaces 50. Alternatively, the computing devices 18 may communicate with one or more networks such as a local area network (LAN), a general wide area network (WAN), and/or a public network (e.g., the network 28 shown in FIG. 1) via a network adapter 52. As depicted, the network adapter 52 may communicate with the other components of the computing device 20 via the bus 36.

As will be appreciated by one skilled in the art, aspects of the disclosed invention may be embodied as a system, method or process, or computer program product. Accordingly, aspects of the disclosed invention may take the form of an entirely hardware embodiment, an entirely software embodiment (including firmware, resident software, microcode, etc.) or an embodiment combining software and hardware aspects that may all generally be referred to herein as a “circuit,” “module,” or “system.” Furthermore, aspects of the disclosed invention may take the form of a computer program product embodied in one or more computer readable media having computer readable program code embodied thereon.

Any combination of one or more computer readable media (for example, storage system 40) may be utilized. In the context of this disclosure, a computer readable storage medium may be any tangible or non-transitory medium that can contain, or store a program (for example, the program product 42) for use by or in connection with an instruction execution system, apparatus, or device. A computer readable storage medium may be, for example, but not limited to, an electronic, magnetic, optical, electromagnetic, infrared, or semiconductor system, apparatus, or device, or any suitable combination of the foregoing.

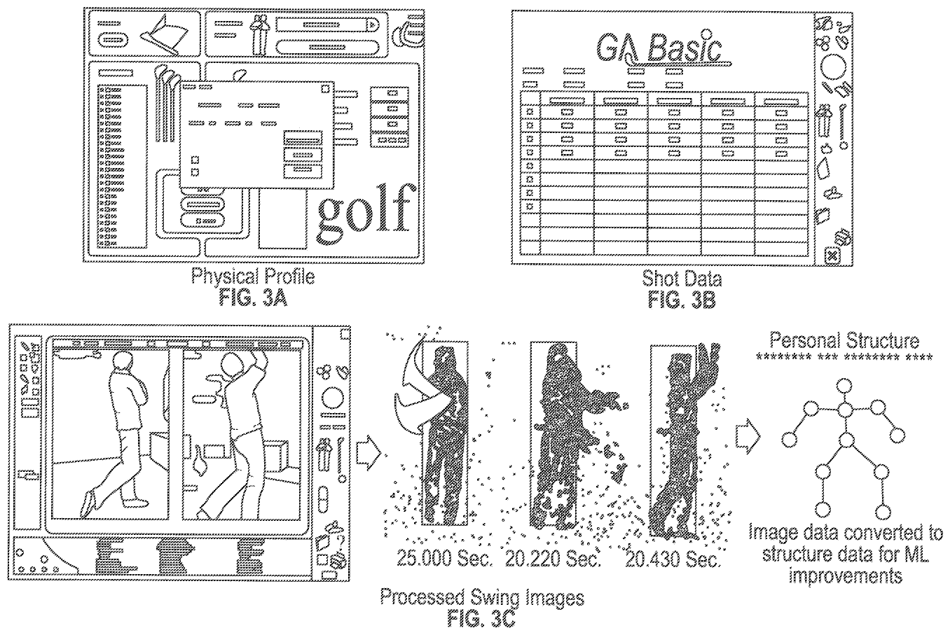

Referring now to FIGS. 1-5, as stated earlier that the machine learning model 24A may be established based on three categories of input data, namely a user's physical profile (FIG. 3A), shot data captured by sensory and imaging platforms 12 (FIG. 3B), and a user's video swing images and relevant postures of the user during the play (FIG. 3C). A user's physical profile may include, but is not limited to: the user's age, height, weight, sex, right/left-handed and the like. The user's physical profile may also include golf club information such as the make and model of the clubs. The shot data captured by sensory and imaging platforms 12 may be similar to the information recorded the sensory and imaging platforms disclosed in U.S. Pat. Nos. 6,302,802 B1 and 10,339,21 B2 both of which are incorporated by reference. This data may include but is not limited to ball flight, ball speed, club direction, club speed as well as other parameters of club swing and ball flight behaviors captured by the sensory and imaging platforms 12. The user's video swing images and relevant postures of the user during the play may be those monitored and recorded by the sensory and imaging platforms 12.

Embodiments of the invention may improve the data capture and processing ability of the sensory and imaging platforms 12 by the additional of the image capturing and processing apparatus. This video image information, along with the shot data information regarding club and ball behavior obtained from the sensory and imaging platforms 12, once captured and processed, may be sent by the sensory and imaging platforms 12, via an appropriate computer and internet connection (which may be wired, wireless, or optical) to the analysis engine 24. The video images and players' postures can be analyzed and determined by the analysis engine 22 in a similar manner as that disclosed in the same inventors' recently granted patent U.S. Pat. No. 10,339,821 B2.

The processed players' swing images may be converted into pictorial body structures of head, arms, limb, waist, legs, and shoulder in pre-selected sequential frames. These identified sequential pictorial body structures may be plotted in graphic forms over a short time period, roughly a fraction of one second, right before and after the club and ball impact. These data and findings may then be compressed in a given format and transmitted to the machine learning model 24A.

The machine learning model 24A may process the information received through a fault detection procedure, and then feed to a machine learning algorithm and model with initial training sets to provide progressively trained response of appropriate play skill correction and improvement suggestions.

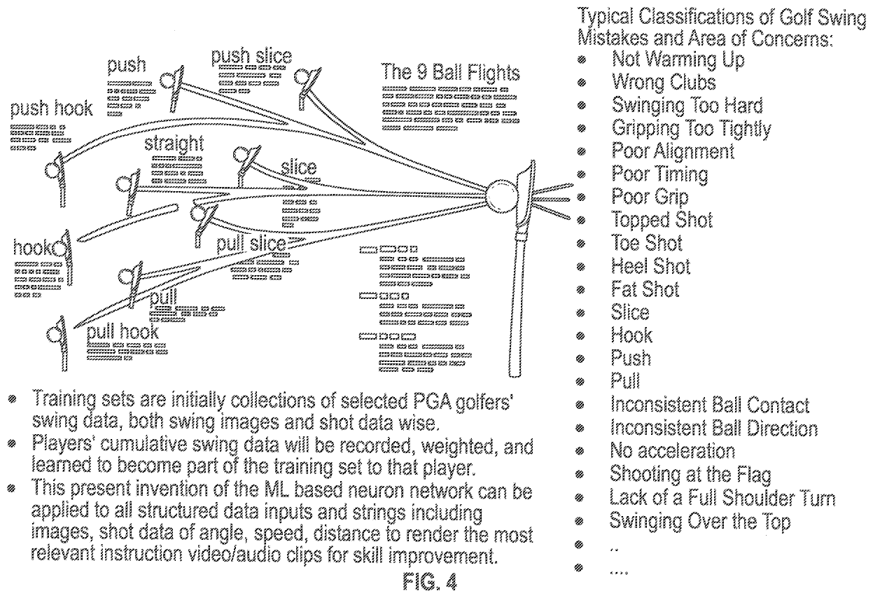

Referring to FIG. 4, an example of nine basic golf ball flight patterns or paths that may be a result of certain classifications of golf swings and commonly made mistakes. The present system 10 may use the machine learning model 24A with initial training sets, which may be derived from selected professional players, to optimize the outcome in a continuous and progressive way of rendering the skill improvement suggestions.

These suggested flight patterns may be categorized into different classifications of swing deficiency and/or players' posture/swing mistakes. The machine learning model 24A may be designed to use a classification predictive modeling. Classification predictive modeling may involve assigning a class label to input examples. The machine learning model 24A may use either binary classification or multi-label classification. Binary classification may refer to predicting one of two classes. Multi-label classification may involve predicting one or more classes for each example.

One such machine learning model may be a neural network. A neural network is a series of algorithms that may be used to recognize underlying relationships in a set of data through a process that mimics the way the human brain operates. In this sense, neural networks refer to systems of neurons, either organic or artificial in nature. Neural networks can adapt to changing input, so the network generates the best possible result without needing to redesign the output criteria. Neural networks may be organized in various layers. The neural network may have an input layer. The input layer may receive the information that is supposed to explain the problem to be analyzed. The neural network may have a hidden layer. The hidden layer may be an intermediate layer allowing neural networks to model nonlinear phenomena. It is said to be “hidden” since there may be no direct contact with the outside world. The outputs of each hidden layer may be used as inputs of the units of the following layer. An output layer may be provided. The output layer may be the last layer of the network. The output layer may be used to produce the result and/or the prediction.

The neural network may come in a variety of configurations. For example, the neural network may come in a variety of configurations such as a fully-connected network, convolutional neural networks, and sequence-based models such as recurrent neural networks (RNN) and long short-term memory (LSTM) models. These models may be parameterized by ‘trainable’ or ‘learnable’ weights that map inputs into a different representational space, usually which is a desired output for regression or classification. These models may be trained with iterative optimization algorithms such as stochastic gradient descent that seek to minimize an appropriate loss function over the data distribution.

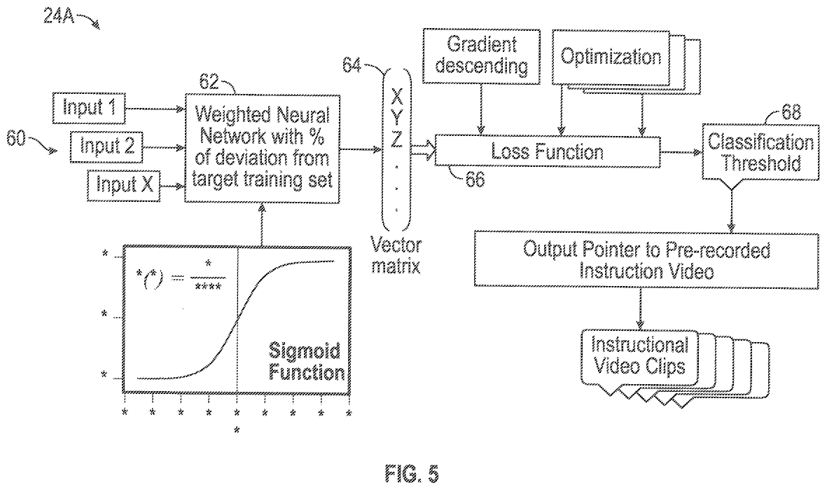

Referring to FIG. 5, a block diagram of the machine learing model 24A may be seen. A set of inputs 60 may be inputted into the weighted neural network 62. The set of inputs may be the user's attributes and captured images as discussed in relation to FIGS. 3A-3C. The neural network 62 may then process these inputs using neurons or, in the case of convolutional neural networks, kernels that apply a linear transformation using the trained weights and biases of the neural network 62. The application of these weights may then be followed by a non-linear activation function such as the sigmoid function or rectified linear unit (ReLU) function that may allow the neural network 62 to uccessfully approximate highly complex data distributions. In the case of a binary classification scheme, a neural network can use a sigmoid function at the output node, with some threshold (usually 0.5) used to make the final classification. This can be extended to a multi-class classification scheme (such as predicting within a pre-determined set of player error types) using a softmax output node.

It is possible that players may be different enough in their physical profiles such as gender, age, height, weight, strength, and skill level, and that the initial training sets as derived from selected PGA players for the machine learning model may not be universally applicable to all players. Thus, it is possible to “fine tune” the base machine learning model for an individual player by accumulating that individual player's swing inputs over time. These machine learning models may be “supervised” using labeled data for classification or “unsupervised” with unlabeled data for cluster output. Of course, given a large enough dataset and sufficient model capacity, it may be possible to have a single model perform reasonably well in providing feedback for a large range of player profiles.

In FIG. 5, each of the inputs 60 including the attributes and images may be captured and fed into the weighted neutral network 62. The neural network 62 then processes these inputs by applying certain kernels or filters and with initially assigned weighted factors such as by adopting Sigmoid function over these inputs and then passes the resulting outcome onto the next layer of neurons. The objective of the neural network 62 is to render and pin point the most relevant and the most accurate determination of the mistakes that a player makes so proper skill improvement suggestions can be made. After the neural network 62, the data may be sent to a related vector and matrix operations 64. For large neural networks, it may be difficult, if not impossible to do a manual calculation or perform loops which may be very inefficient. Thus, the related vector and matrix operations 64 may be used to compress the calculations into a simpler notation. The related vector and matrix operations 64 may be used to provide weighting to certain values as well.

The output of the related vector and matrix operations 64 may be sent to an associated a loss function 66. The loss function 66 may be used to optimize the parameter values in the neural network 62. The loss function 66 may map a set of parameter values for the neural network 62 onto a scalar value that indicates how well those parameters accomplish the task the neural network 62 may be intended to do.

The output of the loss function 66 may be sent to a classification threshold 68. The classification threshold 68 may be used to set a cutoff value of the output from the loss function 66. If the output of the loss function 66 is greater than the classification threshold 68, it may be chosen to point to the pre-recorded instruction video or videos to improve players' skill accordingly.

The quality of the decisions made by the machine learning model 24A may depend on the data and the operating conditions during deployment. Often, operating conditions such as class distribution and misclassification costs may change during the time since the model was trained and evaluated. When deploying a binary classifier that outputs scores, once one knows the new class distribution and the new cost ratio between false positives and false negatives, there are several methods in the literature to help us choose an appropriate threshold for the classifier's scores.

Since the users may be very different in their physical profiles such as gender, age, height, weight, strength, and skill level, and the initial training sets as derived from selected PGA players for the machine learning model may not be universally applied to all players. Thus, the model may need to be “individually trained” for an individual player by accumulating and collecting that individual player's swing inputs over time. These progressively accumulated shot data and swing image inputs may provide the base of adjusting the “weighting factors” of the initial training sets for that individual player. This machine learning model can be either “supervised” with labeled data for classification output or “unsupervised” with unlabeled data for cluster output.

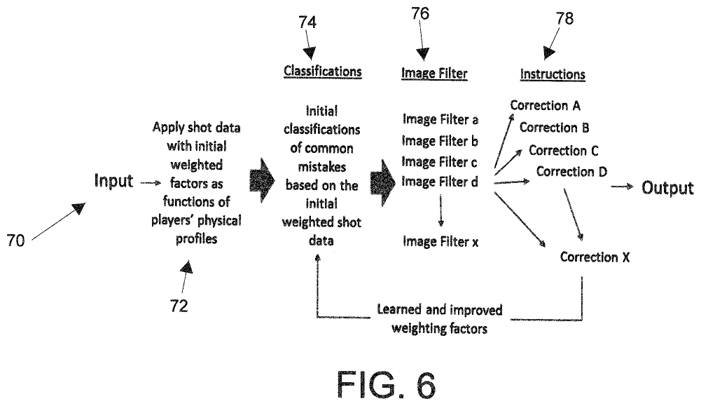

Referring to FIG. 6, in accordance with one or more embodiments of the invention, steps for implementing the learning process may be seen. The user's data may be inputted as shown in 70. An initial classification of common mistakes is determined by weighted shot data and players' physical profiles as shown in 72. Once the initial classification of common mistakes is determined by weighted shot data and players' physical profiles as shown in 74, the player's swing images are used to filter through the initial classifications for consistency and possible re-classifications as shown in 76. Next, the final correction instruction is chosen with feedback of “the individually learned” weighting factors to further improve the initial prediction accuracy as may be seen in 78.

Other than the prediction of any single swing and shot of a specific user, the accumulated history of swing patterns and shot data grouping can also be classified and used as additional inputs to quantify the skill improvement process. Random forest of multiple decision trees approach may be used to cross check and improve the accuracy of pin pointing the prediction accuracy for the desired skill improvement output.

The foregoing description is illustrative of particular embodiments of the application, but is not meant to be a limitation upon the practice thereof. The following claims, including all equivalents thereof, are intended to define the scope of the application.

What is claimed is:

1. A method for generating feedback to a user practicing a skill, comprising:
providing a local platform for acquiring physical parameter data pertaining to motion and position of the user and motion and position of a golf club and a golf ball struck by the golf club during a golf swing by the user;
transmitting via a network at least a portion of the physical parameter data of the motion and position of the golf club and the golf ball struck by the golf club during the golf swing and the physical parameter data associated with the motion and position of the user during the golf swing from the local platform to a machine learning analysis engine as input information;
entering the input information into a machine learning model, the machine learning model having a set of rules and statistical techniques to learn patterns from the input data, and a model which is trained by using evolving training sets, wherein an initial training sets is formed from selected professional golf players physical and swing characteristics and are classified and used to train the machine learning model, and resulting learned weighting factors are feedback and used to refine a model prediction, the machine learning model determining a user's skill deficiencies and providing correction suggestions; and
providing the correction suggestion to the user.

2. The method of claim 1, wherein transmitting the physical parameter data associated with the motion and position of the user during the golf swing from the local platform to a machine learning analysis engine as input information comprises converting a user's swing images into pictorial body structures of head, arms, limb, waist, legs, and shoulder of the user in pre-selected sequential frames, wherein these pre-selected sequential frames may be plotted in graphic forms over a predetermined timeframe.

3. The method of claim 1, wherein the initial training sets is formed from accumulating a user's swing inputs over a predetermined timeframe when the selected professional golf players physical and swing characteristics differs from the user's swing characteristics above a predetermined set point.

4. The method of claim 1, wherein providing a local platform comprises providing a sensory and imaging platform having light or free space signal sources, physical sensing devices, video/image capturing devices, and a local computing device.

5. The method of claim 1, wherein entering the input information into the machine learning model comprises entering a user's physical profile, shot data captured by the local platform and a user's video swing images and postures of the user during the swing captured by the local platform.

6. ethod of claim 1, wherein providing the correction suggestion to the user comprises searching a database for structured skill improvement instructions.

7. The method of claim 6, wherein the structured skill improvement instructions are one of texts, graphs, audios, videos, or combinations thereof.

8. The method of claim 1, comprising:
uploading a plurality of golf flight patterns related to different classifications of swing deficiency and user posture/swing errors; and
linking one of the selected professional players to the user to render the skill improvement suggestions.

9. The method of claim 8, wherein the swing deficiency and user posture/swing errors are given different weighted values.

10. A method for generating feedback to a user practicing a skill, comprising:
providing a local platform for acquiring physical parameter data pertaining to motion and position of the user and motion and position of a golf club and a golf ball struck by the golf club during a golf swing by the user;
transmitting via a network the physical parameter data of the motion and position of the golf club and the golf ball struck by the golf club during the golf swing and the physical parameter data associated with the motion and position of the user during the golf swing recorded by the local platform to a machine learning analysis engine as input information; and
entering the input information into a machine learning model, the machine learning model having a set of rules and statistical techniques to learn patterns from the input data, and a model which is trained by using evolving training sets, wherein an initial training sets is formed from selected professional golf players physical and swing characteristics and are classified and used to train the machine learning model, and resulting learned weighting factors are feedback and used to refine a model prediction, the machine learning model determining a user's skill deficiencies and providing correction suggestions.

11. The method of claim 10, wherein transmitting the physical parameter data associated with the motion and position of the user during the golf swing from the local platform to a machine learning analysis engine as input information comprises converting a user's swing images into pictorial body structures of head, arms, limb, waist, legs, and shoulder of the user in pre-selected sequential frames, wherein these pre-selected sequential frames may be plotted in graphic forms over a predetermined timeframe.

12. The method of claim 10, wherein the initial training sets is formed from accumulating a user's swing inputs over a predetermined timeframe when the selected professional golf players physical and swing characteristics differs from the user's swing characteristics above a predetermined set point.

13. The method of claim 10, wherein providing a local platform comprises providing a sensory and imaging platform having light or free space signal sources, physical sensing devices, video/image capturing devices, and a local computing device.

14. The method of claim 10, wherein entering the input information into the machine learning model comprises entering a user's physical profile, shot data captured by the local platform and a user's video swing images and postures of the user during the swing captured by the local platform.

15. The method of claim 10, comprising providing the correction suggestion to the user.

16. The method of claim 15, wherein the correction suggestions are one of texts, graphs, audios, videos, or combinations thereof.

17. The method of claim 10, comprising:
uploading a plurality of golf flight patterns related to different classifications of swing deficiency and user posture/swing errors; and
linking one of the selected professional players to the user to render the skill improvement suggestions.

18. The method of claim 17, wherein the swing deficiency and user posture/swing errors are given different weighted values.
